(19) United States
(12) Patent Application Publication
(10) Pub. No.: US 2010/0068184 A1
(43) Pub. Date: Mar. 18, 2010

(54) LIVING BODY HEALING ACCELERATOR

(75) Inventors: Naoyuki Moriya, Tokyo (JP); Yukiko Moriya, Tokyo (JP); Koji Kubota, Tokyo (JP)

Correspondence Address: SMITH, GAMBRELL & RUSSELL, 1130 CONNECTICUT AVENUE, N.W., SUITE 1130, WASHINGTON, DC, 20036, US

(73) Assignee: AUREO CO., LTD., Tokyo (JP)

(21) Appl. No.: 12/513,063

(22) PCT Filed: Oct. 22, 2007
(86) PCT No.: PCT/JP2007/070523

§ 371 Date: Apr. 30, 2009

(30) Foreign Application Priority Data
Nov. 2, 2006 (JP) 2006-298478

(51) Int. Cl. A61K 35/66

(52) U.S. Cl. 424/93.3

(57) ABSTRACT

The present invention provides a living body healing, having an accelerating activity in the regeneration ability relating to the cure of damages of cells or tissues caused by toxicity of a drug or a wound in a living body, having high safety, and also not causing any side effect even administered for a long period of time. Problems are solved by a living body healing accelerator containing as an active constituent a mixture of a culture solution obtained after cultivation of a microorganism belonging to the Aureobasidium sp. and mycelia of the microorganism cultivated in the culture solution. The living body healing accelerator of the present invention is preferable used for reducing side effects of an anticancer drug, preferably used for acceleration of healing of a wound, and in addition, preferably used for acceleration of normalization of a blood glucose level and for alleviating a pathological state of leukemia.

### BACKGROUND OF THE INVENTION

1. Field of the Invention

The present invention relates to a living body healing accelerator containing as an active constituent a mixture of a culture solution obtained after cultivation of a microorganism belonging to the Aureobasidium sp. and mycelia of the microorganism cultivated in the culture solution.

2. Description of the Related Art

A culture solution obtained after cultivation of a microorganism belonging to the Aureobasidium sp. is now used as a food or beverage product or as a food additive. The culture solution contains β-glucan, and it is known that the β-glucan is 1,3,1,6-β-D-glucan having a glucose residue with β1,3 linkage as a principal chain and a glucose residue with β-1,6 linkage coupled thereto as a side chain (or a branched chain). (Acta Chemica Scandinavia 17, 1351-1356 (1963) and Agric. Biol. Chem. 47(6), 1167-1172 (1983))

JP 2002-204687 A discloses the fact that a culture solution obtained after cultivation of a microorganism belonging to the Aureobasidium sp. shows high anti-tumor activity and high immunostimulation activity when orally administered and can be used as a preventive drug or a curative drug to various types of diseases.

However, it has not been reported yet that the mixture of the culture solution obtained after cultivation of a microorganism belonging to the Aureobasidium sp. and mycelia of the microorganism cultivated in the culture solution exhibits an effect on cells and tissues for enhancing their regeneration ability.

JP 2002-204687 A also discloses the fact that the culture solution obtained after cultivation of a microorganism belonging to the Aureobasidium sp. can be used as a preventive or curative drug for various types of diseases, and lists diabetes as an example of the diseases. However, JP 2002-204687 A does not disclose any testal data suggesting that the culture solution is active in prevention or therapeutic effect of diabetes.

JP 2002-204687 A also describes that the culture solution obtained after cultivation of a microorganism belonging to the Aureobasidium sp. is effective in suppression of proliferation of leukemia cells in an test system using cultured cells, but does not describe any testal data indicating the pharmaceutical effect of the culture solution based on a result of tests with animals.

Any drug has effects and side effects. When a drug has high effects and low side effects, it is generally considered that the drug has “high safety”. However, there are not few drugs, such as anticancer drugs or steroid drugs, which one has to administer even though the drug has relatively strong side effects. This fact reflects a limit of drugs currently available that the toxicity and the effectiveness cannot safely be separated from each other. In such situation, reduction of toxicity of each drug is vitally significant issue.

For instance, in a case of anticancer drugs, even when cells or tissues are damaged due to expression of toxicities such as the activity for suppression of bone morrow, hepatoxicity, and nephrotoxicity and the drug can not be administered more, sometimes a desirable effect may be obtained by continuing administration of the drug a little more. In other words, if toxicity of the anticancer drug can be reduced in such situation, it would be possible to continue administration of the drug in the state where the effectiveness is exhibited to the maximum extent. This is a compelling need in the clinical field where one's life or death must be determined.

Dissection of skin or mucosa performed in association with a surgical operation and injuries caused by burning or laceration can also be regarded as damage to cells or tissues. When dissection is performed, various preventive treatments are performed, such as prevention of infectious diseases caused by bacteria by oral administration of an antibiotic or direct application of the antibiotic drug to an external wound, reduction of inflammation by administration of an anti-inflammatory drug, or reduction of pain by administration of an analgetic.

The medical treatment for a wound of skin or mucosa caused by a surgical operation aims only reduction of secondary disturbances such as infection of bacteria, inflammation, and pain. Currently healing of the wound is dependent on skin regeneration ability of the living body's. Because of such circumstances, a long time is required until recovery, and there are still concerns about the possibility of side effects caused by administration of drugs.

To solve the problems described above, active efforts have been made for development of a drug capable of accelerating the ability of healing that a living body originally has, but there is still not any drug that can sufficiently satisfy all of the requirements in the effect, safety, production cost, and the like.

As described above, there is the strong need for providing a living body healing accelerator capable of accelerating healing of damages to tissues caused by toxicity of an administered drug or a wound such as a burn in the clinical field.

There is also the strong need for providing a living body healing accelerator which is capable of enhancing a living body's ability of healing for lowering a blood glucose level to a normal value when the level becomes too high due to such diseases as diabetes or the like.

Further, there is also the need for providing a living body healing accelerator for treatment of leukemia causing no side effect even when administered for a long period of time.

### SUMMARY OF THE INVENTION

An object of the present invention is to provide, for solving the problems so ever, a living body healing accelerator which is capable of accelerating regeneration ability of a living body, leading to healing of damages of cells or tissues caused by toxicity of a drug or a wound, having high safety, and also not causing any side effect even administered for a long period of time.

To solve the problems described above, the inventors of the present invention devotes themselves to find out natural materials originated from organisms which has high safety to a human body and are present in abundance in nature as natural resources. During the research process, the inventors found that (1) a mixture of a culture solution obtained after cultivation of a microorganism belonging to the Aureobasidium sp. and mycelia of the microorganism cultivated in the culture solution shows an accelerating activity in the regeneration ability relating to the cure of damages of cells or tissues caused by toxicity of a drug or a wound in a living body, that (2) the mixture shows the activity of suppressing rise of a blood glucose level caused by diabetes, and that (3) the mixture gives the life prolongation effect to a mouse with leukemia-originated cancer cells implanted therein. Based on the facts described above, the inventors found that the mixture is useful as a living body healing accelerator, and completed the present invention.

According to an embodiment of the present invention, provided is a living body healing accelerator including as an active constituent a mixture of a culture solution obtained after cultivation of a microorganism belonging to the Aureobasidium sp. and mycelia of the microorganism cultivated in the culture solution.

According to another embodiment of the present invention, provided is a use of a mixture of a culture solution obtained after cultivation of a microorganism belonging to the Aureobasidium sp. and mycelia of the microorganism cultivated in the culture solution in manufacturing a medicament for acceleration of healing a living body.

According to still another embodiment of the present invention, provided is a method of accelerating healing of a living body, including administering an effective amount of a mixture of a culture solution obtained after cultivation of a microorganism belonging to the Aureobasidium sp. and mycelia of the microorganism cultivated in the culture solution to human or an animal in need thereof for accelerating healing of the living body.

According to a preferred embodiment of the present invention, the living body healing accelerator is used for reducing side effects of an anticancer drug. In this case, the anticancer drug is more preferably 5-fluorouracil (5-FU).

According to another preferred embodiment of the present invention, the living body healing accelerator is used for acceleration of healing of a wound. In this case, the wound is caused in association with a surgical operation or by burn on skin, and the living body healing accelerator is for being more preferably directly applied to an affected area.

According to still another preferred embodiment of the present invention, the living body healing accelerator is used for normalization of a blood glucose level.

According to still further another preferred embodiment of the present invention, the living body healing accelerator is used for improving a pathological state of leukemia.

Further, in the present invention, the microorganism belonging to the Aureobasidium sp. is preferably the Aureobasidium pullulans strain M-1 (Deposited number of FERM BP-08615 at International Patent Organism Depositary, National Institute of Advanced Industrial Science and Technology (Independent Administrative Corporation)) or the Aureobasidium pullulans strain M-2 (Deposited number of FERM BP-10014 at International Patent Organism Depositary, National Institute of Advanced Industrial Science and Technology (Independent Administrative Corporation)).

With the present invention, it is possible to provide a living body healing accelerator containing as an active constituent a mixture of a culture solution obtained after cultivation of a microorganism belonging to the Aureobasidium sp. and mycelia of the microorganism cultivated in the culture solution, and side effects of anticancer drugs can be reduced when the living body healing accelerator is administered concurrently.

Further, acceleration in healing of a wound such as a burn can be performed by applying the living body healing accelerator to the wound

Still further, acceleration in normalization of a blood glucose level in a patient suffering from hyperglycemia can be performed by administering the living body healing accelerator into the patient.

A pathological state of leukemia can be improved by administering the living body healing accelerator.

As described above, with the living body healing accelerator according to the present invention, it is possible to accelerate activity in the regeneration ability relating to healing of damages of skin and tissues in a living body.

### BRIEF DESCRIPTION OF THE DRAWINGS

FIG. 1 is a view showing change in a volume of a tumor;

FIG. 2 is a view showing change in a body weight;

FIG. 3 is a view showing change in the number of leukocytes;

FIG. 4 is a view illustrating the effect of a Aureobasidium culture solution on reduction of a weight of an organ;

FIG. 5 is a view illustrating a result of comparison of erythema areas in 3 days after cautery treatment;

FIG. 6 is a view illustrating a result of comparison between blood glucose levels before and after administration of the test sample; and

FIG. 7 is a view showing an effect on a survival rate of mice carrying cancer cells originated from leukemia.

### DETAILED DESCRIPTION OF THE INVENTION

The present invention is described in detail below.

The term of “microorganism belonging to the Aureobasidium sp.” as used herein is applicable to any strain separated from the environment (such as food products, soil, or indoor environment).

The most preferable example of the microorganism belonging to the Aureobasidium sp. is strains belonging to Aureobasidium pullulans. More specifically, the preferable microorganisms include strains stocked as single strains respectively such as IFO 4464, IFO 4466, IFO 6353, IFO 7757, ATCC 9348, ATCC 3092, ATCC 42023, and ATCC 433023. Especially, the Aureobasidium pullulans strain M-1 (Deposited number of FERM BP-08615 at International Patent Organism Depositary, National Institute of Advanced Industrial Science and Technology (Independent Administrative Corporation)) is the most preferable one because of the high yield of 1,3,1,6-β-D-glucan.

It is also possible to use a mutated strain, which is prepared by subjecting a stock strain separated as a single strain to the ordinary technology for mutation. The ordinary technology for mutation as used herein include, but not limited to UV radiation, or chemical processing with such chemicals as nitrosoguanidine, ethidium bromide, ethyl methanesulfonate, or sodium nitrite, and especially the Aureobasidium pullulans strain M-2 (Deposited number of FERM BP-10014 at International Patent Organism Depositary, National Institute of Advanced Industrial Science and Technology (Independent Administrative Corporation)) is the most preferable one because of the high yield of 1,3,1,6-β-D-glucan, and also because accumulation of color in a culture solution is lower as compared to the parent strain M-1.

“A mixture of a culture solution obtained after cultivation of a microorganism belonging to the Aureobasidium sp. and mycelia of the microorganism cultivated in the culture solution” according to the present invention is obtained by culturing microorganisms belonging to the Aureobasidium sp. The microorganism belonging to the Aureobasidium sp. can be cultured according to any of known methods (Refer to, for instance, Japanese Patent Laid-open Publication No. Sho 57-149301). That is, the culture may require inoculating bacteria in a culture medium (with the pH of 5.2 to 6.0) containing a 0.5 to 1.0 mass % of carbon source (such as sucrose), 0.1 mass % of a nitrogen source, and extremely small amounts of other substances (such as vitamins, or inorganic materials), and cultivating the bacteria for 2 to 3 days at a temperature in the range from 20 to 30° C. in the aerated state, preferably in the aerated state with agitation. When β-1,3-1,6-gulcan produces, viscosity of the culture solution becomes higher, and a gel-like material having high viscosity is generated. The culture solution obtained as described above generally contains 0.6 to 1.8 mass % of a solid phase, and 5 to 80 mass % of the β-1,3-1,6-gulcan is contained in the solid phase. In the present invention, it is preferable to use a culture containing 1 mass % or more of β-1,3-1,6-gulcan of the solid phase, and is more preferable to use a culture containing 5 mass % or more of β-1,3-1,6-gulcan in the solid phase. When a concentration of β-1,3-1,6-gulcan in the culture is too low, the sufficient physiological effect of the glucan can not be expected.

Quantification of β-1,3-1,6-gulcan can be performed according to the method described in Japanese Examined Patent Publication No. Hei 3-48201. That is, after culturing is finished, the culture solution is sterilized and is subjected to centrifugation to remove the fungus bodies. Then a mixture solution of chloroform and butanol is added in the obtained solution by 10 volume % and the resultant mixture solution is shaked (sevage method) and then to centrifugation to remove chloroform and insoluble matters. The operating sequence is repeated twice, and then the solution is subjected to ethanol precipitation to recover precipitated materials. The recovered materials are dissolved in distilled water, and pullulan is decomposed by processing with an enzyme. Then the decomposed materials are subjected to dialysis in distilled water, and the dialysis solution is subjected to ethanol precipitation to recover the precipitated material (β-1,3-1,6-gulcan) and determine the yield.

In the present invention, the culture solution obtained as described above may be used after sterilized by heating or pressurizing, or may be used after condensed or dried according to the necessity. Culture of bacteria belonging to Aureobasidium sp. are used as food additives like a thickening stabilizer, and the safety is high.

When the mixture of the culture solution obtained after cultivation of the microorganism belonging to the Aureobasidium sp. and mycelia of the microorganism cultivated in the culture solution is condensed, the condensation is preferably performed at low temperature in the depressurized state. The condensation may be performed until the mixture is dried and solidified. In this case, it is more preferably to perform freeze drying or spray drying in the depressurized state. It is to be noted that the mixture may be filtered before condensation and then condensed.

The “mixture of the culture solution obtained after cultivation of the microorganism belonging to the Aureobasidium sp. and mycelia of the microorganism cultivated in the culture solution” according to the present invention may be further purified. Chromatography or elution using an ion exchange resin may be employed singly or in combination for purifying the mixture according to the present invention.

When chromatography is employed, any of normal phase chromatography, reversed phase chromatography, high-performance liquid chromatography, centrifugal liquid chromatography, column chromatography, thin-layer chromatography and the like may be used singly or in combination. Conditions for purification, such as a carrier and a solvent for elution may be selected according to the necessity in correspondence to the selected chromatography. For instance, in a case of normal phase chromatography, a chloroform methanol-based solvent may be used, and in reversed phase chromatography, a water-methanol-based solvent may be used.

As the elution method using an ion exchange resin, for instance, the method may be employed in which the eluted liquid is diluted and dissolved in water or lower alcohol, the solution is contacted to the ion exchange resin to be adsorbed therein and then eluted with water or lower alcohol. The lower alcohol used in this method is as described above, and methanol is especially preferable. There is no specific restriction over the ion exchange resin to be used in this method on the condition that the ion exchange resin can be used for purification in this technical field, and for instance, any of a porous and cross-linked polystyrene-based resin having a large mesh-like structure, urbanlite, cellulose, and the like may be used.

The “living body healing accelerator” according to the present invention is used for accelerating regeneration of cells and tissues damaged by a drug or a wound. The uses include, for instance, acceleration of regeneration of cells or tissues damaged by side effects of an anticancer drug, such as suppression of bone morrow, hepatoxicity, and nephrotoxicity, and acceleration of healing of a tissue damaged by a wound caused by dissection of skin or mucosa performed in association with a surgical operation, burn, laceration, or the like. Further, the living body healing accelerator according to the present invention is also effective in accelerating normalization of a blood glucose level in a hyperglycermia patient. In addition, the healing accelerator is also effective in improvement of the pathological state of a patient suffering from leukemia.

The “living body healing accelerator” according to the present invention may be orally administered, and also may be administered as an external preparation which is applied to skin or the like. When the living body healing accelerator is used for reducing side effects of an anticancer drug, for accelerating normalization of a blood glucose level, or for improving a pathological state of leukemia, preferably the healing accelerator according to the present invention is administered orally. When the drug is used for accelerating healing of a tissue damaged by a wound caused by dissection of skin or mucosa performed in association with a surgical operation, burn, or laceration, the living body healing accelerator is preferably directly applied to the affected area.

The “living body healing accelerator” according to the present invention may be sold as it is as a product in the market, but generally various types of constituents for improving the taste or for being formed into a required form are added and blended therein, and further, a flavor is added to prepare a final product.

The constituents added and blended in the “living body healing accelerator” according to the present invention include various types of sugars, emulsifying agents, sweeteners, acidifiers, fruit juices or the like. More specifically, sugars such as glucose, sucrose, fructose, honey; sugar alcohols such as sorbitol, xylitol, erythritol, lactitol, and palatinit; and emulsifying agents such as sucrose fatty acid ester, glycerin sugar fatty acid ester, and lecithin may be used as the constituents. In addition, various types of vitamins such as vitamin A, vitamin B, vitamin C, and vitamin E, or extracts of herb, constituents of cereals, constituents of vegetables, and constituents of milk may be added and blended therein to obtain living body healing accelerator having an excellent taste.

The flavors which may be added to the “living body healing accelerator” according to the present invention include those based on yogurt, berries, oranges, Chinese quince, Japanese basil, citrus, apple, mint, grape, pear, custard cream, peach, melon, banana, tropical, herb, black tea, coffee, and the like, and the flavors may be used singly or in combination. There is no specific restriction over an amount of the added flavor, and the amount of added flavor is preferably in the range from 0.05 to 0.5 mass %, and more preferably in the range from about 0.1 to about 0.3 mass % for providing a good taste.

The “living body healing accelerator” described above can be processed into market products in any states including a solid state, a liquid state, and the like.

The “living body healing accelerator” according to the present invention can be used as various forms of preparations such as granules, tablets, or capsules prepared by any known technology in the field of drug and food production, together with medicinal acceptable salts, diluents, preserving agents, coloring agents, and taste-adjusting agents.

Further, the “living body healing accelerator” according to the present invention can be used for health food. The health food means foods especially aimed for maintaining and promoting welfare and health more positively as compared to ordinary foods. More specifically, the foods include, liquid, semisolid or solid products, such as cookie, rice cracker, jelly, sweet jelly of beans, yogurt, steamed bread, other types of cakes, cold beverage, energy drink, soup, and the like. In addition, the “living body healing accelerator” according to the present invention may be decocted to be a tea form. Further, the living body healing accelerator according to the present invention can be processed into health foods by being added, by means of mixing, applying, or spraying, during the final stage of the production process or into the final products.

A dosage of the “living body healing accelerator” according to the present invention varies according to age, symptom, and other factors of a recipient. For instance, when orally administered, one dosage for an adult is 3 to about 100 g when used in the gel-like state, and 200 mg to about 3 g when used in the powder state. When used as an extract, the dosage is in the range from 50 mg to about 2 g according to a level of purification or a moisture content, and in any case, the accelerator is preferably administered 3 times a day and about 30 minutes before each meal. When used as a health food, an amount of the material to be used does not affect a taste or an appearance of the food, and for instance, when used in the powder state, the dosage is preferably in the range from 1 to about 100 g against 1 kg of the food.

Further, the “living body healing accelerator” according to the present invention can be used as an external preparation for skin in various product forms such as lotion (cosmetic water), creams or the like for cosmetics, emulsions, cosmetic water, pack drugs, skin milk (emulsion), gel, powder, lip creams, lip rouge, under makeup, foundation, sunblock creams, bath agents, body shampoos, body rinse agents, soap, cleansing foam, ointments, patches, jelly-like preparations, and aerosol agents.

When the “living body healing accelerator” according to the present invention is used as an external preparation for skin, various constituents or additives as described below, which are generally used in cosmetics, quasi drugs, and drugs, may be blended therein according to the necessity.

That is, the various constituents or additives which may be blended in the living body healing accelerator according to the present invention include, but not limited to, moisturizing agents such as glycerin, vaseline, urea, hyaluronic acid, heparin; ultraviolet absorbers or scattering agents such as PABA derivatives (para-aminobenzoic acid, Escalora 507, or the like), cinnamic acid derivatives (such as neoheliopan, parsol MCX, and sunguard B), salicylic acid derivatives (such as octyl salicylate), benzophenone derivatives (such as ASL-24, ASL-24S), dibenzoylmethane derivatives (such as Parsol A, Parsol DAM), heterocyclic derivatives (tinuvin-based one or the like), titanium oxide; metal chelators such as disodium edetate, trisodium edetate, citric acid, sodium citrate, tartaric acid, sodium tartrate, lactic acid, malic acid, sodium polyphosphate, sodium methaphosphate, and gluconic acid; sebum-preventing agents such as salicylic acid, sulfur, caffeine, tannin; germicides or disinfectants such as benzarconium chlorate, benzethonium chlorate, and chlorhexidine gluconate; anti-inflammatory agents such as diphenhydramine chlorate, tranexamic acid, guaiazulene, azulene, allantoin, hinokitol, glycyrrhizic acid and salts thereof, glycyrrhizic acid derivatives, and glycyrrhetenic acid; vitamins such as vitamin A, vitamin B group (B1, B2, B6, B12, and B15), folic acid, nicotine acids, pantothenic acids, biotin, vitamin C, vitamin D group (D2, D3), vitamin E, ubiquinones, vitamin K (K1, K2, K3, and K4); amino acids and derivatives thereof, such as asparagine acid, glutamic acid, alanine, lysine, glycine, glutamine, serine, cysteine, cystine, tyrosine, proline, arginine, and pyrrolidone carbonate; whitening agents such as retinol, tocopherol acetate, magnesium ascorbyl-phosphate, ascorbic acid glucoside, arbutin, kojic acid, ellagic acid, and placental extract fluids; antioxidants such as butylhydroxytoluene, butylhydroxyanisole, and propyl gallate; astringent drugs such as zinc chloride, zinc sulfate, zinc phenol, zinc oxide, and aluminate-potassium sulfate; sugars such as glucose, fructose, maltose, sucrose, trehalose, erythritol, mannitol, xylitol, and lactitol; and essences from various plants such as licorice root, matricaria, horse chestnut, strawberry geranium, paeoniae radix, Chinese quince, Scutellaria root, cork tree bark, coptis rhizome, Houttuyniae Herba, and maidenhair tree. In addition, oily substances, surfactants, thickifiers, alcohols, powder substances, coloring agents, and the like may be added in the living body healing accelerator according to the present invention depending on the necessity.

When the “living body healing accelerator” according to the present invention is used as an external preparation for skin, the application quantity is preferably in the range from 10 to about 1,000 mg/cm² in a case of a gel and in the range from 1 to about 50 mg/cm² in a case of powder. In a case of the extract, the quantity is preferably in the range from 1 to 500 mg/m² according to a degree of purification, a moisture content, or the like.

### EXAMPLES

The present invention is described below in further detail with reference to examples thereof, but it is to be noted that the present invention is not restricted by the examples in any sense.

In the tests described below, a mixture of a culture solution prepared by culturing the Aureobasidium pullulans strain M-2 according to the known method and mycelia therein (with the solid phase contents of 1.5 to 2 mass %; hereinafter referred to as Aureobasidium culture solution) was used.

### Example 1

### Test for Reduction of Side Effects of Anticancer Drugs

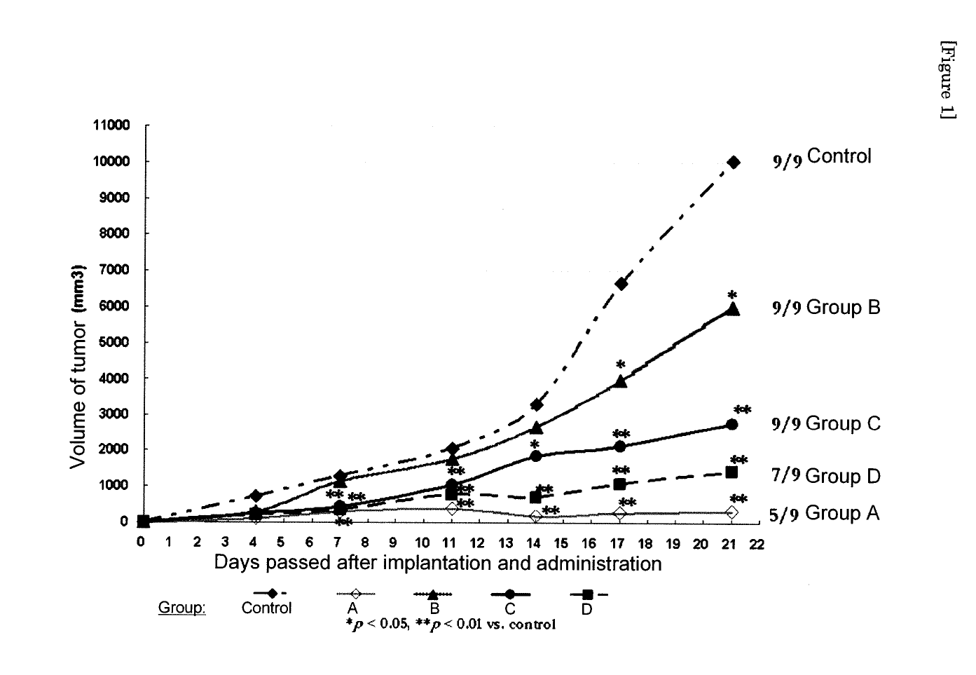

In the test, an anticancer drug 5-FU (20 mg/kg/day) and/or a culture solution of Aureobasidium sp. (2 g/kg to 30 g/kg/day) are administered to mice with sarcoma implanted therein. A body weight, the number of leukocytes, and a weight of organs of each mouse are measured as indicators of side effects and obtained values are compared to each other. Immediately after sarcoma is implanted, each test sample is orally administered by having each test animal freely drink the sample as drinking water from a bottle for water absorption. Table 1 shows allocation of the test animals and the average daily dose of the anticancer drug and/or culture solution of the Aureobasidium sp. The results of the test are shown in FIG. 1 to FIG. 4.

TABLE 1
Average doses of the anticancer drug and/or the Aureobasidium
culture solution

| | | Number of | Average dose |
|---|---|---|---|
| Test group | Tested substance | animals (n) | (mg/kg) |
| Control | Physiological saline | 9 | — |
| group | | | |
| Group A | Anticancer drug | 9 | 19.7 ± 2.7 |
| | (5-FU) | | |
| Group B | Aureobasidium culture | 9 | 28,700 ± 1,700 |
| | solution | | (430.5 ± 22.5)*¹ |
| Group C | Anticancer drug | 9 | 19.0 ± 1.6 + |
| | (5-FU) + Aureobasidium | | 1,900 ± 200 |
| | culture solution | | (28.5 ± 2.5)*¹ |
| Group D | Anticancer drug | 9 | 13.5 ± 1.1 + |
| | (5-FU) + Aureobasidium | | 13,500 ± 1,100 |
| | culture solution | | (202.4 ± 16.3)*¹ |

*¹Converted score of solid phase content (1.5%) (mg/kg)

Result 1 (Effect of Avoiding Toxic Death by Anticancer Drugs)

The ordinate axis of the FIG. 1 represents volumes of tumors and the abscissa axis represents the number of days after start of the administration of the anticancer drugs. Allocation of test groups is the same as that showed in Table 1. As seen from the data for the group A, a volume of the tumor was significantly restrained by treatment with the anticancer drugs as compared to the result of the control group not having been subjected to any specific treatment. However, in 21 days after start of the treatment with the ant-cancer drugs, 4 out of 9 mice in the test A group in which treatment with the anticancer drug treatment was carried out were dead. On the other hand, there was no example of death in the control group although growth of a tumor was observed.

The above result obviously indicates the characteristics of the treatment with an anticancer drug, and in this type of treatment, the anticancer drug can suppress growth of a tumor, but normal cells are damaged by toxicity of the anticancer drug. This negative aspect spoils the positive result of the treatment with an anticancer drug. However, when the Aureobasidium culture solution (2 g/kg/day) was used concurrently in the test C group, tumor growth was significantly suppressed (p<0.01), and no test animal dies like in the control group. The dose of 2 g/kg/day corresponds to 30 mg/kg/day in administration with respect to the solid content (1.5%). It means that the Aureobasidium culture solution shows the effect when administered with almost same level of dosage as the anticancer drug. Further, in the test B group in which treatment was carried out only with the Aureobasidium culture solution (29 g/kg/day), the statistically significant anticancer effect (p<0.05) was confirmed without the death of any test animal.

Result 2 (Effect of Mitigating Reduction of Body Weight Associated with Administration of an Anticancer Drug)

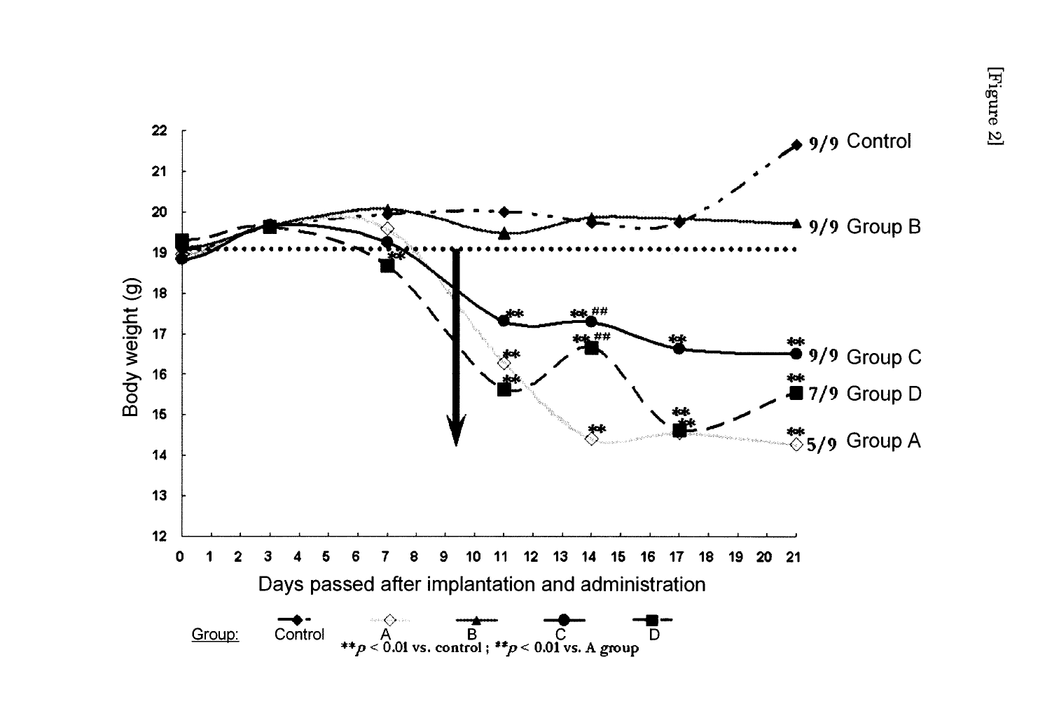

The ordinate axis in FIG. 2 represents the body weight, while the abscissa axis represents the number of days passed after administration of an anticancer drug is started. Allocation of the animal groups is the same as that shown in Table 1. As seen from the data for the test A group, the side effects by treatment with the anticancer drug appeared with reduction of 26% body weight. However, in the test C group in which the Aureobasidium culture solution was used concurrently in treatment with an anticancer drug (by about 2 g/kg/day), the reduction of body weight due to treatment with the anticancer drug could be mitigated to 13%. The mitigation in reduction of body weight caused by side effects of administration of the anticancer drug was recognized as statistically significant different (p<0.05) as compared to a case where treatment was carried out only with the anticancer drug.

Result 3 (Effect of Mitigating Reduction of Leukocytes Associated with Administration of an Anticancer Drug)

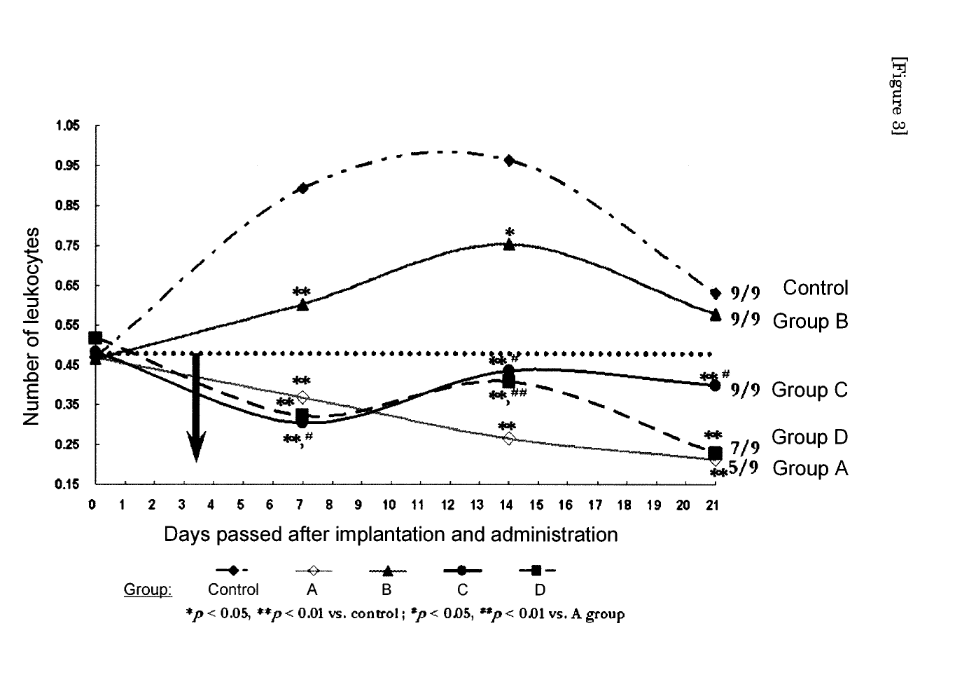

The ordinate axis in FIG. 3 represents the number of leukocytes, while the abscissa axis represents the number of days passed after administration of an anticancer drug is started. Allocation of the animal groups is the same as that shown in Table 1. As seen in the data for the test A group, the side effects by treatment with the anticancer drug appeared with reduction of 42% of the number of leukocytes. However, in the test C group in which the Aureobasidium culture solution was used concurrently in treatment with an anticancer drug (by about 2 g/kg/day), reduction of leukocytes continues for 7 days after start of the treatment, but then the tendency for recovery appeared, and the reduction of leukocytes could be mitigated to 16% in 21 days after start of the treatment. The mitigation in reduction of leukocytes caused by side effects of administration of the anticancer drug was recognized as statistically significant different (p<0.05) as compared to a case where treatment was carried out only with the anticancer drug.

Result 4: (Effect of Mitigating Atrophy of Organs Caused by Administration of an Anticancer Drug)

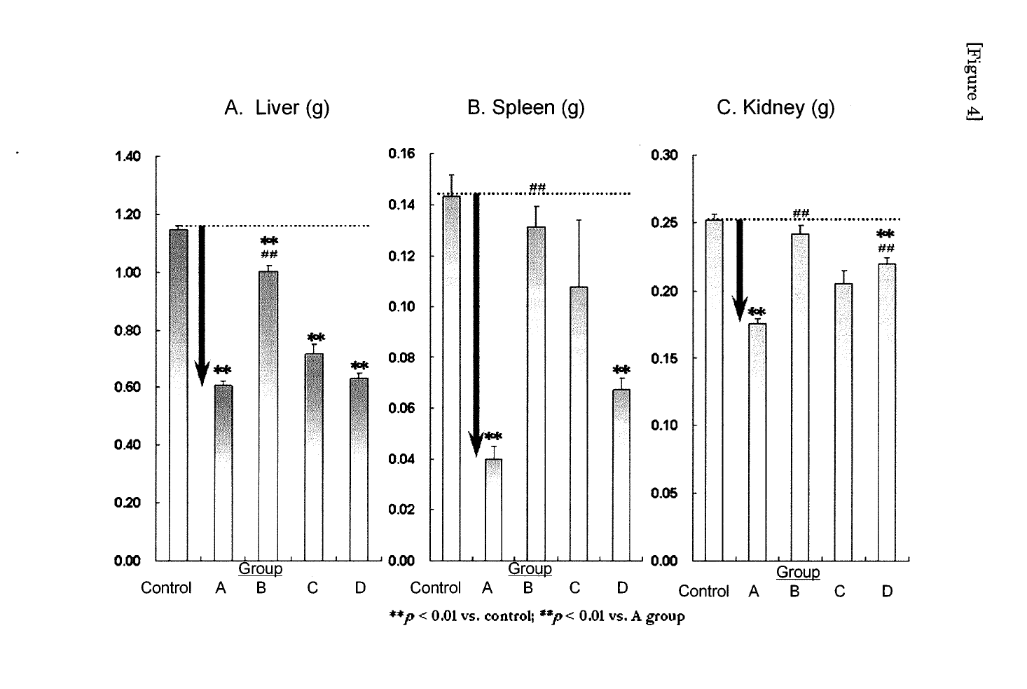

The ordinate axis in FIG. 4 represents weights of organs, while the ordinate axis represents a result of measurement of weights of organs, that is, livers, spleens, and kidneys in each test group in 21 days after start of administration of an anticancer drug. Allocation of the test groups is the same as that shown in Table 1. As seen from the data for the test A group, the side effect of reduction in weights of organs caused in association with treatment with an anticancer drug appeared with reduction of 48% in the livers, of 72% in the spleens, and of 28% in the kidneys. Atrophy of the spleen, which may be regarded as a storage site for lymphocytes, is especially remarkable, and this phenomenon is closely related to the reduction of leukocytes described in Result 3. In the test C group and the test D group, in which the Aureobasidium culture solution was administered concurrently with an anticancer drug, reduction in weights of organs associated with treatment with the anticancer drug could be mitigated.

It is confirmed, based on the result of measurement as described above, that the Aureobasidium culture solution reduces side effects of an anticancer drug and can enhance effects of treatment with the anticancer drug without causing death due to toxicity of the anticancer drug.

### Example 2

### Test for Acceleration of Healing of Burn

In the test, burn was prepared by burning moxa at specific regions called Kyokuchi of both arms of 12 test subjects respectively (age of each test subject is as shown in Table 2), the culture solution of Aureobasidium sp. was applied to the burn on the left arm to compare influence of the effects over healing of the burn with that on the right arm which was not subjected to any specific treatment.

More specifically, moxa was burnt on the regions called Kyokuchi on both arms of each test subject to prepare burn, and then a patch of absorbent cotton having the size of 1.5 cm×1.2 cm with 1.5 mL of Aureobasidium culture solution absorbed therein was adhered to the site of the left arm where moxa was burnt and fixed with adhesive tapes, and the absorbent cotton patch was removed when the cotton was dried (in 4 to 5 hours). The treatment was performed once a day for three days. On the other hand, not specific treatment was carried out to the burn on the right arm. A erythema caused by the burn was photographed every day, and subjective symptoms concerning pain, burning feeling, blister, and itch were inquired to each test subject.

TABLE 2
Background of test subjects

| | | Number of Examples |
|---|---|---|
| Item | Classification | (%) |
| Gender | Male | 8 (66.7) |
| | Female | 4 (33.3) |
| Age | 30 to less than 40 | 3 (25.0) |
| | 40 to less than 50 | 5 (41.7) |
| | 50 to less than 60 | 2 (16.7) |
| | 60 to less than 70 | 1 (8.3) |
| | 70 to less than 80 | 1 (8.3) |

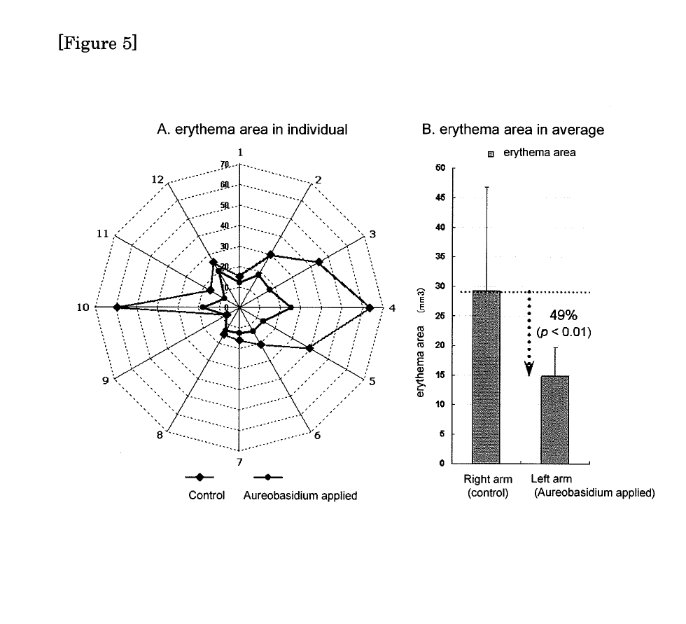

A result in 3 days after application of moxa is as shown in FIG. 5. FIG. 5A shows values of erythema areas on each test subject, while FIG. 5B shows the average values. A remarkable difference is observed on each of the test subjects No. 3, No. 4, No. 5, and No. 10, and it was confirmed that, on the left arm of each test subject to which the Aureobasidium culture solution was applied, the erythema area was reduced to 51% of that on the right arm not subjected to any specific treatment with the statistically significant difference (p<0.01).

This result suggest that healing of the burn was accelerated by application of the Aureobasidium culture solution. The conceivable mechanism seems to be attributed to acceleration of regeneration of cells and tissues, and the effect emerges as accelerated regeneration of a tissue damaged due to toxicity of an anticancer drug or acceleration in healing of a burn.

Based on the results as described above, it could be confirmed that the Aureobasidium culture solution reduces side effects of anticancer drugs and also accelerates healing of a burn.

### Example 3

### Test for Accelerating in Normalization of a Blood Glucose Level

The test was performed by using mice, in each of which a diabetes was induced by streptozotocin, and also by comparing blood glucose levels before and after administration of test samples. Allocation of the tested animals is as shown in Table 3 below. In Table 3, the “normal group” indicates a blood glucose level in the normal state where treatment with streptozotocin is not performed. On the other hand, the “negative control group” indicates a blood glucose level in a mouse in which diabetes was generated by being subjected to treatment with streptozotocin. The “positive control group” indicates a blood glucose level when tolbutamide which was an accelerator of insulin secretion (200 mg/kg) was administered to a mouse carrying diabetes caused by streptozotocin. The “test A group” and “test B group” each indicates a blood glucose level when each of the Aureobasidium culture solution (2 g/kg and 10 g/kg) was administered to a mouse carrying diabetes caused by streptozotocin. It is to be noted that each sample was intrapenitoreally administered.

TABLE 3

| | Mouse carrying | | |
|---|---|---|---|
| | diabetes | | Aureobasidium |
| | caused by | | culture |
| Test group | streptozotocin | Tolubutamide | solution |
| Normal group | no | | |
| Negative control | yes | | |
| group | | | |
| Positive control | yes | 200 mg/kg | |
| group | | (intrapenitoreally) | |
| Test A group | yes | | 2 g/kg |
| | | | (intrapenitoreally) |
| Test B group | yes | | 10 g/kg |
| | | | (intrapenitoreally) |

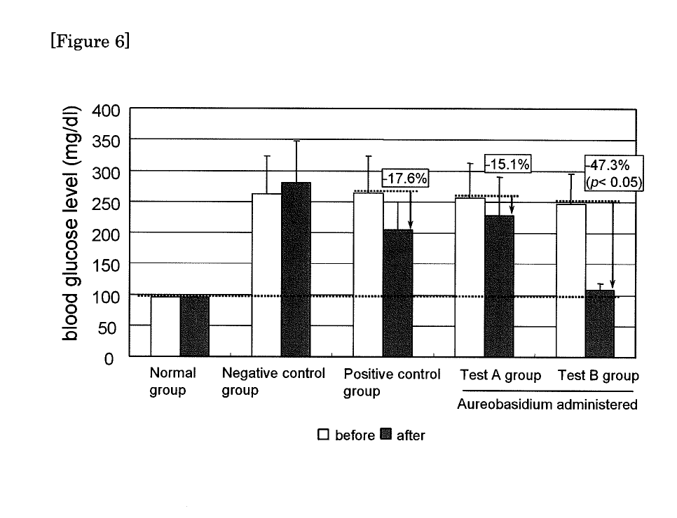

A result of the test is shown in FIG. 6. FIG. 6 is plotted with the ordinate axis indicating a blood glucose level and with the abscissa axis indicating treatment performed to each group of the tested mice. A left bar in each group represents an average value of blood glucose levels before administration of the test sample, while a right bar represent an average value of blood glucose levels after administration of the test sample.

As clearly understood from FIG. 6, it is recognized that the blood glucose level drops in the dose-dependent mode when the Aureobasidium culture solution is administered. The percentage of descent of blood glucose level was 15.1% when the Aureobasidium culture solution was administered at a dose of 2 g/kg (test A group in FIG. 6), and 47.3% when the Aureobasidium culture solution was administered at a dose of 10 g/kg (test B group in FIG. 6). In the test B group in which the Aureobasidium culture solution was administered at the applied dose of 10 g/kg, the blood glucose level decreased as compared to that before administration with a statistically significant difference (p<0.05), and because of the decrease, the blood glucose level was controlled to the normal level. On the other hand, a descent percentage of a blood glucose level was 17.4% in the positive control group in which tolbutamide was administered at a dose of 200 mg/kg, which clearly indicates that the more remarkable effect of descending the blood glucose level is obtained in the case where the Aureobasidium culture solution is administered at a dose of 10 g/kg. This applied dose of 10 g/kg corresponds to 150 mg/kg (in which 32% of β-glucan was contained) with the conversion of a solid phase (1.5%). Therefore, the possibility was suggested that active constituents effective equal to or more than tolbutamide are included in the Aureobasidium culture solution.

### Example 4

### Anti-Leukemia Test

The test was carried out according to the ordinary method by using mice each with the leukemia-originated tumor cell P388 implanted therein and also by comparing survival rates of the mice after the test samples were administered. Just after the tumor was implanted, each test sample was orally administered by mixing the sample in water in a bottle for drinking water and making the mice freely drink the water. Table 4 shows allocation of the tested animals and average daily applied doses of anticancer drugs, lactic acid bacteria product name: “EC-12”, produced by Combi Corporation), and Aureobasidium culture solution. 10 mice were used for one test sample group.

TABLE 4
Average applied doses of anticancer drugs, Aureobasidium
culture solution, and/or lactic acid bacteria

| | | Number of | Average dose |
|---|---|---|---|
| Test group | Tested substance | animals (n) | (mg/kg) |
| Control | Glycerin fatty acid ester | 10 | — |
| group | solution | | |
| Group A | Anticancer drug (5-FU) | 10 | 2.0 ± 0.18 |
| Group B | Aureobasidium culture | 10 | 4,862 ± 625 |
| | solution | | (72.9 ± 9.4)*¹ |
| Group C | Aureobasidium culture | 10 | 6,977 ± 1,023 |
| | solution + lactic acid | | (104.7 ± 15.4) + |
| | bacteria | | 17.4 ± 2.6 |
| Group D | Anticancer drug (5-FU) + | 10 | 1.8 ± 0.37 + |
| | Aureobasidium culture | | 5,607 ± 1,126 |
| | solution | | (84.1 ± 16.9)*¹ |
| Group E | Anticancer drug (5-FU) + | 10 | 2.0 ± 0.19 + |
| | Aureobasidium culture | | 6,150 ± 593 |
| | solution + lactic acid | | (92.3 ± 8.9)*¹ + |
| | bacteria | | 15.4 ± 1.5 |

*¹Converted score of solid phase content (1.5%) (mg/kg)

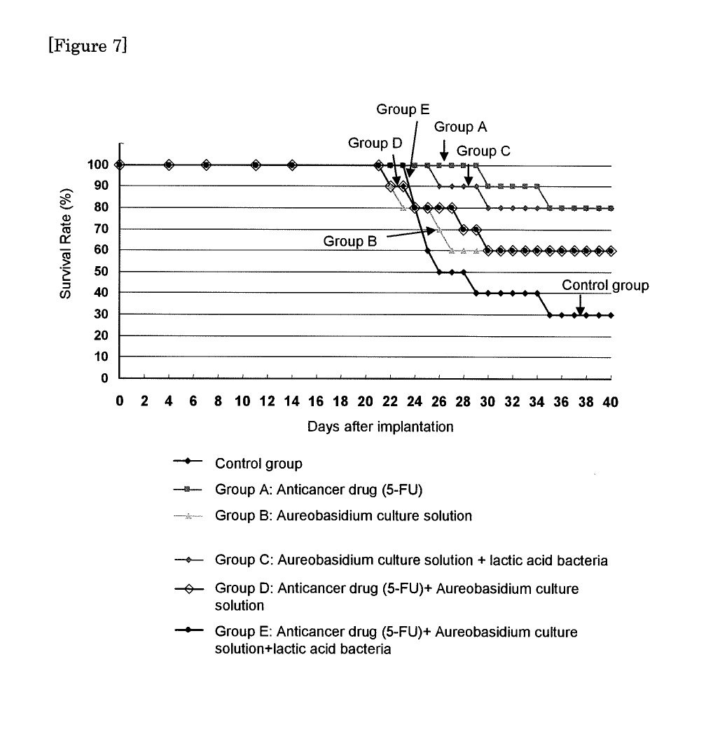

A result of this test is shown in FIG. 7.

As clearly seen from FIG. 7, a result of comparison of survival rates indicates that the survival rate was only 30% in the group in which only the solvent of 0.05% glycerin fatty acid ester was administered, but that the survival rate in 40 days after implantation was improved to 60% in the group (group B) in which the Aureobasidium culture solution was administered. Further, in the group in which the Aureobasidium culture solution and the lactic acid bacteria were administered concurrently (group C), the survival rate in 40 days after implantation was improved up to the same level as that (80%) in the group in which the anticancer drug was administered (group A). It was confirmed based on this result that the Aureobasidium culture solution was effective for anti-leukemia. Further it was suggested that the effect was enhanced by concurrently administering the lactic acid bacteria together with the Aureobasidium culture solution. There were some reports on the effect of concurrent use of lactic acid bacteria with the Aureobasidium culture solution, and the result of the test conducted by the inventor on an anti-leukemia effect supports the reports.

On the other hand, in the case where an anticancer drug and the Aureobasidium culture solution were administered concurrently (group D), and in the case where all of the anticancer drug, Aureobasidium culture solution, and lactic acid bacteria were concurrently administered (group E), only the survival rate in the case where only the Aureobasidium culture solution was administered (group B) could be obtained.

### INDUSTRIAL APPLICABILITY

It was confirmed that, by using the Aureobasidium culture solution according to the present invention, side effects of anticancer drugs can be reduced, that healing of a wound caused by burn can be accelerated, that normalization of a blood glucose level can be accelerated, and that life of a mouse with leukemia-originated cancer cells implanted therein can be prolonged.

Because the Aureobasidium culture solution according to the present invention can reduce toxicity of an anticancer drug, administration of the drug can be continued in the state where the efficiency is exhibited to the maximum level. Therefore it can be said that the Aureobasidium culture solution is very useful in the clinical field where determination for a life-or-death matter must be made.

It may also be said that acceleration of healing of tissues damaged, for instance, by a burn is very useful for maintaining social activities of the burnt person.

Further, because the Aureobasidium culture solution according to the present invention can accelerate normalization of a blood glucose level, it can be said that the matter is very useful in preventing or reducing complications caused by hyperglycemia.

Further, because no side effect occurs even when administered for a long period of time, it can be said that the Aureobasidium culture solution according to the present invention is very useful in improving a pathological state of leukemia.

1. A living body healing accelerator comprising as an active constituent a mixture of a culture solution obtained after cultivation of a microorganism belonging to the Aureobasidium sp. and mycelia of the microorganism cultivated in the culture solution.

2. A living body healing accelerator according to claim 1, wherein the living body healing accelerator is used for reducing a side effect of an anticancer drug.

3. A living body healing accelerator according to claim 2, wherein the anticancer drug is 5-fluorouracil (5-FU).

4. A living body healing accelerator according to claim 1, wherein the living body healing accelerator is used for acceleration of healing of a wound.

5. A living body healing accelerator according to claim 4, wherein the wound is caused in association with a surgical operation or by burn on skin, and the living body healing accelerator is for being directly applied to an affected area.

6. The living body healing accelerator according to claim 1, wherein the living body healing accelerator is used for accelerating normalization of a blood glucose level.

7. A living body healing accelerator according to claim 1, wherein the living body healing accelerator is used for improving a pathological state of leukemia.

8. A living body healing accelerator according to any one of claims 1 to 7, wherein the microorganism belonging to the Aureobasidium sp. is the Aureobasidium pullulans strain M-1 (Deposited number of FERM BP-08615 at International Patent Organism Depositary, National Institute of Advanced Industrial Science and Technology (Independent Administrative Corporation)) or the Aureobasidium pullulans strain M-2 (Deposited number of FERM BP-10014 at International Patent Organism Depositary, National Institute of Advanced Industrial Science and Technology (Independent Administrative Corporation)).

9. A use of a mixture of a culture solution obtained after cultivation of a microorganism belonging to the Aureobasidium sp. and mycelia of the microorganism cultivated in the culture solution in manufacturing a medicament for acceleration of healing a living body.

10. A use according to claim 9, wherein the medicament is for reducing a side effect of an anticancer drug.

11. A use according to claim 10, wherein the anticancer drug is 5-fluorouracil (5-FU).

12. A use according to claim 9, wherein the medicament is for acceleration of healing of a wound.

13. A use according to claim 12, wherein the wound is caused in association with a surgical operation or by burn on skin, and the medicament is for being directly applied to an affected area.

14. A use according to claim 9, wherein the medicament is for accelerating normalization of a blood glucose level.

15. A use according to claim 9, wherein the medicament is for improving a pathological state of leukemia.

16. A use according to any one of claims 9 to 15, wherein the microorganism belonging to the Aureobasidium sp. is the Aureobasidium pullulans strain M-1 (Deposited number of FERM BP-08615 at International Patent Organism Depositary, National Institute of Advanced Industrial Science and Technology (Independent Administrative Corporation)) or the Aureobasidium pullulans strain M-2 (Deposited number of FERM BP-10014 at International Patent Organism Depositary, National Institute of Advanced Industrial Science and Technology (Independent Administrative Corporation))

17. A method of accelerating healing of a living body, comprising
administering an effective amount of a mixture of a culture solution obtained after cultivation of a microorganism belonging to the Aureobasidium sp. and mycelia of the microorganism cultivated in the culture solution to human or an animal in need thereof for accelerating healing of the living body.

18. A method of accelerating healing of a living body according to claim 17, wherein the mixture is administered to human or an animal in need thereof to whom an anticancer drug is administered for reducing the side effect of the anticancer drug.

19. A method of accelerating healing of a living body according to claim 18, wherein the anticancer drug is 5-fluorouracil (5-FU).

20. A method of accelerating healing of a living body according to claim 17, wherein the mixture is administered to human or an animal in need thereof who is wounded for accelerating healing of the wound.

21. A method of accelerating healing of a living body according to claim 20, wherein the wound is caused in association with a surgical operation or by burn on skin, and the mixture is for being directly applied to an affected area.

22. A method of accelerating healing of a living body according to claim 17, wherein the mixture is administered to human or an animal in need thereof who has abnormal blood glucose level for accelerating normalization of the blood glucose level.

23. A method of accelerating healing of a living body according to claim 17, wherein the mixture is administered to human or an animal in need thereof for alleviating a pathological state of leukemia.

24. A method of accelerating healing of a living body according to claim 17, wherein the microorganism belonging to the Aureobasidium sp. is the Aureobasidium pullulans strain M-1 (Deposited number of FERM BP-08615 at International Patent Organism Depositary, National Institute of Advanced Industrial Science and Technology (Independent Administrative Corporation)) or the Aureobasidium pullulans strain M-2 (Deposited number of FERM BP-10014 at International Patent Organism Depositary, National Institute of Advanced Industrial Science and Technology (Independent Administrative Corporation)).
